(19) United States
(12) Patent Application Publication
(10) Pub. No.: US 2018/0267011 A1
(43) Pub. Date: Sep. 20, 2018

(54) METHOD OF PREDICTING DEFORMATION OF RESIN MOLDED ARTICLE

(71) Applicant: AISIN SEIKI KABUSHIKI KAISHA, Kariya-shi (JP)

(72) Inventors: Mie Funamoto, Anjo-shi (JP); Tsuyoshi Tanigaki, Nagoya-shi (JP); Daisuke Kaneda, Nishio-shi (JP); Takuro Matsunaga, Nagakute-shi (JP); Kenzo Fukumori, Nagakute-shi (JP)

(73) Assignee: AISIN SEIKI KABUSHIKI KAISHA, Kariya-shi (JP)

(21) Appl. No.: 15/924,899

(22) Filed: Mar. 19, 2018

(30) Foreign Application Priority Data
Mar. 17, 2017 (JP) 2017-053426
Feb. 19, 2018 (JP) 2018-026642

(51) Int. Cl. G01N 33/44; G01N 25/00; G06F 17/50

(52) U.S. Cl. CPC G01N 33/442; G06F 2217/12; G06F 17/5086; G01N 25/00

(57) ABSTRACT

A method of predicting deformation of a resin molded article includes: a step of acquiring resin temperature distribution data at the time of forming the resin molded article; a step of creating crystallinity distribution data, corresponding to the resin temperature distribution data, based on a first correlation between a temperature and crystallinity of the resin molded article, which is obtained using an actually measured crystallinity of the resin molded article; a step of creating mechanical property value distribution data, corresponding to the crystallinity distribution data, based on a second correlation between the crystallinity and the mechanical property value of the resin molded article, which is obtained from the actually measured crystallinity and the mechanical property value of the resin molded article; and a step of predicting the deformation of the resin molded article using the resin temperature distribution data and the mechanical property value distribution data.

### CROSS REFERENCE TO RELATED APPLICATIONS

This application is based on and claims priority under 35 U.S.C. § 119 to Japanese Patent Application 2017-053426, filed on Mar. 17, 2017, and Japanese Patent Application 2018-026642, filed on Feb. 19, 2018, the entire contents of which are incorporated herein by reference.

### TECHNICAL FIELD

This disclosure relates to a method of predicting deformation of a resin molded article.

### BACKGROUND DISCUSSION

When designing a mold for resin molding, a computer simulation analysis may be performed to predict deformation (deformation amount or deformation state) of a resin molded article taken out from the mold. When the accuracy of prediction of the deformation of the resin molded article by this computer simulation analysis is low, the number of prototyping times of the mold increases, which results in an increase in the manufacturing costs of the mold. Therefore, it is necessary to improve the accuracy of prediction of the deformation of the resin molded article in such analysis.

JP 2002-219739 A (Reference 1) discloses a method of predicting deformation of a resin molded article including a step of creating a model in which each shell of a shell model of the resin molded article, which is formed of a crystalline resin, is divided into a plurality of layers in the thickness direction thereof, a step of predicting the crystallinity of the resin for each layer of each shell, a step of obtaining a linear expansion coefficient in a flow direction of the resin and a linear expansion coefficient in a direction orthogonal to the flow direction of the resin for each layer of each shell from the predicted crystallinity, and a step of predicting a deformation amount of the resin molded article after releasing the resin molded article using the obtained linear expansion coefficients. According to Reference 1, by using the model in which each shell is divided in the thickness direction and by predicting the linear expansion coefficients both in the flow direction of the resin and the direction orthogonal thereto, it is possible to improve prediction accuracy even in the case where the deformation of the resin molded article is predicted using a two-dimensional shell model.

JP 09-262887 A (Reference 2) discloses a method in which the PVT curve of a resin and the specific volume of the resin depending on crystallization behavior at the time of molding are calculated based on the PVT characteristics of the resin at an arbitrary crystallinity, and the shrinkage rate of the resin is predicted therefrom. According to Reference 2, since it is possible to calculate the shrinkage rate conforming to the crystallinity at the time of molding, prediction accuracy can be improved by predicting deformation of a resin molded article by using the predicted shrinkage rate.

JP 09-230008 A (Reference 3) discloses a method in which a shrinkage rate in an in-plane direction and a shrinkage rate in a thickness direction are obtained from an equation representing the shrinkage anisotropy of a resin, and warpage deformation of a resin molded article is predicted using the obtained shrinkage rates. According to Reference 3, it is possible to improve prediction accuracy by predicting the warpage deformation of the resin molded article in consideration of the shrinkage anisotropy of the resin.

In a method of predicting deformation of a resin molded article known in the related art, particularly, in a method of predicting deformation of a resin molded article using a non-fiber-reinforced resin, the linear expansion coefficient of a resin may be input as distribution data. However, due to the influence of molding conditions or the like, it is difficult to accurately predict mechanical property values of the resin and to demonstrate the predicted value. For this reason, in many cases, no mechanical property value is used, or mechanical property values are given as constant values (fixed values). However, in practice, it is considered that the mechanical property values of a resin molded article is not constant, but differs depending on molding regions. In other words, it is considered that the mechanical property values of a resin molded article have a distribution.

The mechanical property values of the resin are involved in the magnitude of deformation of the resin molded article. In particular, when the resin molded article is formed of a non-reinforced material that does not contain reinforcing fibers (that is not reinforced with fibers), the mechanical property values of the resin greatly contribute to the magnitude of deformation of the resin molded article. Therefore, in the prediction of the deformation of the resin molded article, the accuracy of prediction of the deformation greatly deteriorates when the mechanical property values of the resin are given by fixed values.

In addition, for the sake of convenience, a method of predicting deformation of a resin molded article by giving mechanical property values as distribution data has also been proposed. For example, JP 2012-152964 A (Reference 4) discloses a deformation prediction method of predicting deformation of a resin molded article by giving a Young's modulus depending on a temperature as distribution data to a shape model. The data on distribution of the Young's modulus illustrated in Reference 4 is considered to be derived from a theoretical equation representing the temperature dependency of the Young's modulus. However, at the time of actual manufacture, the mechanical property values such as, for example, the Young's modulus is less likely to be derived with good accuracy only from the theoretical equation relating to the temperature, and thus, prediction accuracy is not sufficiently improved even when the distribution of such theoretically calculated mechanical property values are given.

Thus, a need exists for a method of predicting deformation of a resin molded article, which is not susceptible to the drawback mentioned above.

### SUMMARY

An aspect of this disclosure provides a deformation prediction method of predicting deformation of a resin molded article, which is resin-molded using a mold, the method including: a resin temperature distribution data acquisition step (S1) of acquiring resin temperature distribution data at the time of forming the resin molded article; a crystallinity distribution data creation step (S2) of creating crystallinity distribution data, which is data on distribution of a crystallinity of the resin molded article corresponding to the resin temperature distribution data, based on a first correlation, which is a correlation between a temperature and crystallinity of the resin molded article and is obtained using an actually measured crystallinity of the resin molded article, which is actually resin-molded using the mold; a mechanical property value distribution data creation step (S3) of creating mechanical property value distribution data, which is data on distribution of a mechanical property value of the resin molded article corresponding to the crystallinity distribution data, based on a second correlation, which is a correlation between the crystallinity and the mechanical property value of the resin molded article and is obtained from the actually measured crystallinity and the mechanical property value of the resin molded article, which is actually resin-molded using the mold; and a deformation prediction step (S4) of predicting the deformation of the resin molded article, which is taken out from the mold and is cooled to a predetermined temperature, using the resin temperature distribution data and the mechanical property value distribution data.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a schematic view illustrating a configuration of an analysis system in which a deformation prediction method according to the present embodiment is performed;

FIG. 2 is a schematic view illustrating a functional configuration of an analysis device;

FIG. 3 is a flowchart schematically illustrating the flow of deformation prediction by a deformation analysis unit;

FIG. 4A is a graph illustrating a correlation between a mold temperature and a crystallinity;

FIG. 4B is a graph illustrating a correlation between a mold temperature and a skin layer thickness;

FIG. 4C is a graph illustrating a correlation between a mold temperature and a core layer thickness;

FIG. 4D is a graph illustrating a correlation between a mold temperature and a crystallinity of a surface area;

FIG. 4E is a graph illustrating a correlation between a mold temperature and a crystallinity of a boundary area;

FIG. 5 is a graph illustrating an actually measured crystallinity distribution;

FIG. 6 is a graph illustrating an actually measured Young's modulus distribution;

FIG. 7 is a graph illustrating a correlation between a crystallinity and a Young's modulus;

FIG. 8 is a view illustrating a mesh division model of a resin molded article, which is divided into five areas; and

FIG. 9 is a graph illustrating an actually measured warpage amount (deformation amount) and a predicted warpage amount (deformation amount).

### DETAILED DESCRIPTION

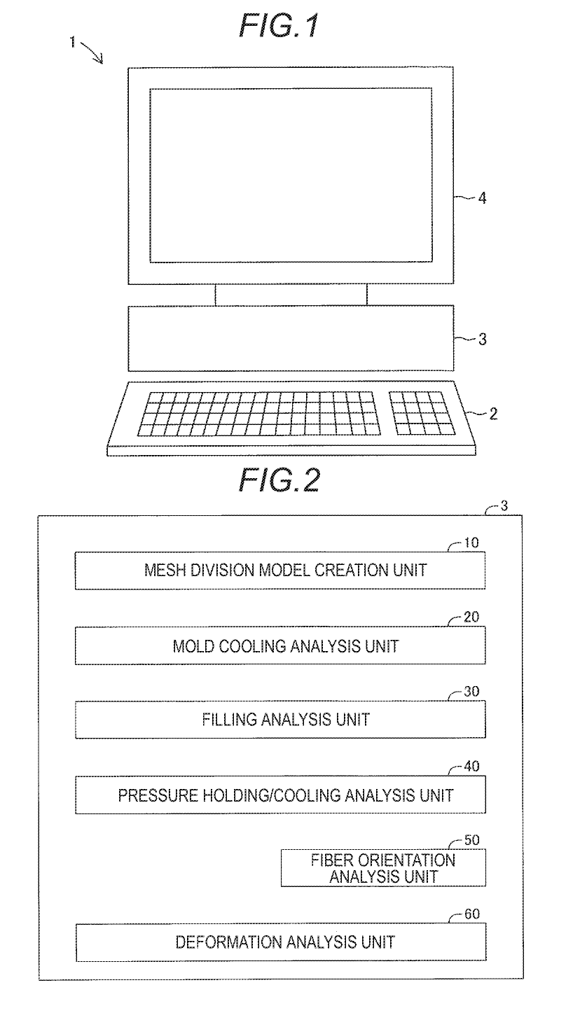

Hereinafter, an embodiment disclosed herein will be described with reference to the drawings. FIG. 1 is a schematic view illustrating a configuration of an analysis system in which a deformation prediction method according to the present embodiment is performed. The analysis system executes an analysis (prediction) to what extent a resin molded article, which is molded by injection molding, which is resin molding using a mold, is deformed when the resin molded article is taken out from the mold and cooled to room temperature, i.e. a deformation analysis by a computer simulation. In addition, in order to perform the deformation analysis, for example, an analysis of a temperature distribution in the mold (mold cooling analysis), a filling analysis of a resin filled in the mold (flow analysis), an analysis of a resin temperature and a resin pressure at the time of pressure holding/cooling executed after the completion of filling (pressure holding/cooling analysis) are also executed. In addition, the resin, which is an analysis target, is a crystalline resin.

As illustrated in FIG. 1, the analysis system 1 according to the present embodiment includes an input device 2, an analysis device 3, and a display device 4. Conditions required for an analysis by the analysis system 1 (the type of resin, molding conditions (e.g., resin temperature, injection time, pressure holding time, holding pressure, and cooling time), and mold temperature conditions (e.g., the type, flow rate, and temperature of cooling water), and a shape model) are input to the input device 2. As the input device 2, for example, a keyboard or a mouse may be exemplified. The analysis device 3 is configured with a microcomputer having, for example, a CPU, a ROM, and a RAM, and executes the above-described analysis (prediction) based on the conditions input from the input device 2. The display device 4 displays the results analyzed (predicted) by the analysis device 3.

FIG. 2 is a schematic view illustrating a functional configuration of the analysis device 3. As illustrated in FIG. 2, the analysis device 3 includes a mesh division model creation unit 10, a mold cooling analysis unit 20, a filling analysis unit 30, a pressure holding/cooling analysis unit 40, a fiber orientation analysis unit 50, and a deformation analysis unit 60. In addition, the fiber orientation analysis unit 50 performs an analysis only when the resin is a fiber-reinforced resin.

The mesh division model creation unit 10 inputs shape data indicating a shape model created by a CAD tool (e.g., shape data of a resin molded article, shape data of a mold used to mold the resin molded article, shape data of a cooling pipe provided in the mold, and shape data of a gate and a runner). Then, the mesh division model creation unit 10 divides a shape indicated by the input shape data into a plurality of meshes. Thus, a shape model, which is divided into a plurality of meshes (hereinafter referred to as a “mesh division model”), is created. The mesh division model corresponds to the element division model according to the aspect of this disclosure.

The mold cooling analysis unit 20 executes the mold cooling analysis. Specifically, the mold cooling analysis unit 20 calculates a predicted value of the temperature of a mold at the time of injection molding for each mesh constituting the mesh division model of the mold based on various conditions input from the input device 2. Thus, data on distribution of the predicted value of the temperature of the mold at the time of resin molding is created.

The filling analysis unit 30 executes the filling analysis. Specifically, the filling analysis unit 30 calculates over time, for example, the filling pattern of a resin injected into the mold and the temperature and pressure of the resin flowing in the mold, based on data on distribution of the predicted value of the temperature of the mold, which is created by the mold cooling analysis unit 20, and the various conditions input from the input device 2. That is, changes in the resin temperature distribution at the time of resin filling are calculated. Then, the filling analysis unit 30 outputs the calculated results to the display device 4. By the filling analysis executed by the filling analysis unit 30, it is possible to predict how the molten resin injected into the mold is filled in the mold, and to predict the temperature distribution and pressure distribution of the resin filled in the mold.

The pressure holding/cooling analysis unit 40 executes the pressure holding/cooling analysis. Specifically, the pressure holding/cooling analysis unit 40 calculates, over time, changes in the temperature and linear expansion coefficient of the resin molded article in the mold for each mesh constituting the mesh division model of the resin molded article, during a period from the start of holding of the pressure on the resin in the mold to the taking out of the resin molded article from the mold through the completion of the cooling of the resin molded article with the mold, based on the data on distribution of the predicted value of the temperature of the mold, which is created by the mold cooling analysis unit 20, the temperature distribution and pressure distribution of the resin in the mold at the time of completion of filling, which are calculated by the filling analysis unit 30, and the various conditions input from the input device 2. Then, the pressure holding/cooling analysis unit 40 creates resin temperature distribution data and linear expansion coefficient distribution data by assigning the calculated temperature and linear expansion coefficient to each mesh constituting the mesh division model of the resin molded article. By the pressure holding/cooling analysis executed by the pressure holding/cooling analysis unit 40, it is possible to predict changes in the temperature and pressure of the resin molded article at the time of pressure holding and at the time of cooling.

The fiber orientation analysis unit 50 predicts the orientation of fibers in the fiber-reinforced resin from the flow of the resin at the time of filling based on the results obtained by the filling analysis unit 30 and the results obtained by the pressure holding/cooling analysis unit 40. By the fiber orientation analysis executed by the fiber orientation analysis unit 50, it is possible to predict a combined effect of the results of physical property values (e.g., temperature and pressure) of the resin by the pressure holding/cooling analysis unit 40 and a fiber orientation. In addition, when the fiber-reinforced resin is not used, the fiber orientation analysis by the fiber orientation analysis unit 50 is not executed.

The deformation analysis unit 60 executes the deformation analysis. Specifically, the deformation analysis unit 60 acquires data on distribution of a resin temperature (hereinafter referred to as “resin temperature distribution data”) created by the pressure holding/cooling analysis unit 40 and data on distribution of a linear expansion coefficient at the time of taking out the resin molded article from the mold. The deformation analysis unit 60 may also acquire, for example, data on distribution of the predicted value of the temperature of the mold at the time of resin molding, which is created by the mold cooling analysis unit 20, and data on distribution of the temperature of the resin flowing in the mold, which is calculated by the filling analysis unit 30. In addition, the deformation analysis unit 60 calculates the deformation amount of the resin molded article at the time when the resin molded article taken out from the mold is cooled to room temperature using the acquired distribution data and Young's modulus distribution data to be described later. Then, the deformation analysis unit 60 outputs data indicating the shape of the deformed resin molded article to the display device 4. By the analysis of the deformation analysis unit 60, it is possible to predict deformation of the resin molded article.

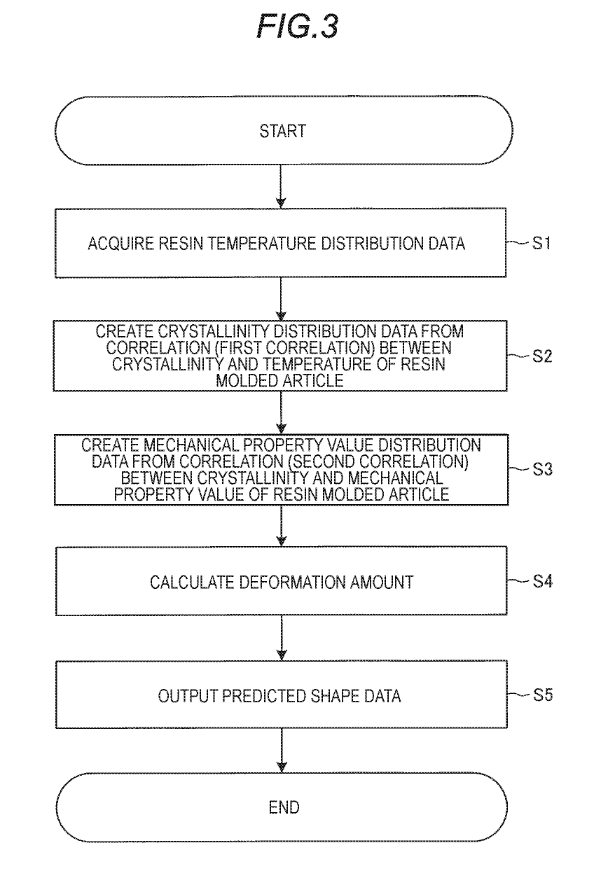

FIG. 3 is a flowchart schematically illustrating the flow of deformation prediction by the deformation analysis unit 60. According to this, in the prediction of deformation of a resin molded article, the deformation analysis unit 60 firstly acquires resin temperature distribution data at the time of molding in step 1 (hereinafter, step is abbreviated as “S”) in FIG. 3 (resin temperature distribution data acquisition step). In addition, the resin temperature distribution data acquired here is created by assigning a predicted value of the resin temperature in a region corresponding to each of a plurality of meshes constituting a mesh division model of the resin molded article, to each of the meshes. In addition, the linear expansion coefficient distribution data is also created by assigning a predicted value of the linear expansion coefficient of the resin molded article in a region corresponding to each of the plurality of meshes constituting the mesh division model of the resin molded article, to each of the meshes. In addition, in the present embodiment, resin temperature distribution data from the start of forming the resin molded article to the taking out of the resin molded article from the mold are used as the resin temperature distribution data at the time of molding. The resin temperature distribution data are calculated over time. Thus, the data acquired in S1 are resin temperature distribution change data.

Subsequently, in S2, the deformation analysis unit 60 creates crystallinity distribution data, which is data on distribution of the crystallinity of the resin molded article corresponding to the resin temperature distribution data (resin temperature distribution change data), based on a correlation (first correlation) between the crystallinity and temperature of the resin molded article, which is derived from a correspondence relationship between an actually measured crystallinity of the resin molded article, which is actually resin-molded (injection-molded) using the same resin as an analysis target resin, and the temperature of the mold used at that time (crystallinity distribution data creation step).

Subsequently, in S3, the deformation analysis unit 60 creates Young's modulus distribution data (mechanical property value distribution data), which is data on distribution of the Young's modulus (mechanical property value) of the resin molded article corresponding to the crystallinity distribution data, based on a correlation (second correlation) between the crystallinity and Young's modulus (mechanical property value) of the resin molded article, which is derived from a correspondence relationship between the actually measured crystallinity and Young's modulus of the resin molded article, which is actually resin-molded using the same resin as the analysis target resin (mechanical property value distribution data creation step).

In the crystallinity distribution data creation step, the crystallinity distribution data is created based on the correlation (first correlation) between the actually measured crystallinity and the temperature of the resin molded article, and in the mechanical property value distribution data creation step, the Young's modulus distribution data is created based on the correlation (second correlation) between the actually measured crystallinity and the actually measured Young's modulus. Hereinafter, a method of deriving these correlations will be described.

### <Derivation of Relative Relationship (First Correlation) Between Crystallinity and Temperature of Resin Molded Article>

Before performing the deformation analysis by the deformation analysis unit 60, a sample of a resin molded article having the same shape (e.g., a flat plate shape) as the shape model of the resin molded article is actually injection-molded using the same resin as the analysis target resin.

In addition, the samples of resin molded articles having a flat plate shape were injection-molded while changing the set temperature Tm of the mold (a fixed type mold and a movable type mold) variously. Thus, the samples of resin molded articles corresponding to the set temperature Tm of a plurality of molds are actually injection-molded. In addition, at a point in time at which cooling of the resin in the mold is completed and the sample of the resin molded article is taken out from the mold, the temperature of the mold is substantially equal to the set temperature. Thus, the set temperature Tm may be said to be the temperature of the mold at the point in time at which the sample of the resin molded article is taken out from the mold. In addition, the respective temperatures of the fixed-type mold and the movable-type mold included in the mold may be set to be different from each other.

Subsequently, the crystallinity in the thickness cross-sectional direction (thickness direction) of a plurality of actually resin-molded samples was measured at the interval of 10 μm. It is very difficult to measure a detailed crystallinity at each extremely minute distance such as the interval of 10 μm. In the present embodiment, the crystallinity was measured using SPring-8 (Hyogo Ken Beamline BL 24 XU), which is a synchrotron radiation facility, and using a synchrotron X-ray scattering method. By this measurement, it is possible to obtain a crystallinity distribution in the thickness direction.

FIG. 5 is a graph schematically illustrating an example of an actually measured crystallinity distribution. In the graph of FIG. 5, the horizontal axis represents the position in the thickness direction of a sample, and the vertical axis represents crystallinity X. In addition, in the graph of FIG. 5, the left end position of the horizontal axis is a surface (fixed side surface), which has been in contact with the fixed type mold, among the surfaces of the sample, and the right end position of the horizontal axis is the surface (movable side surface) which has been in contact with the movable type mold, among the surfaces of the sample. In addition, in the example illustrated in FIG. 5, the set temperature of the fixed type mold is 40° C., and the set temperature of the movable type mold is 90° C.

As illustrated in FIG. 5, it can be seen that the crystallinity distribution exists in the thickness direction of the sample. In addition, the crystallinity in the vicinity of both the surfaces of the sample, i.e. the surfaces, which have been in contact with the mold, is low, and the crystallinity in the central portion in the thickness direction is high. The crystallinity in the central portion of the sample in the thickness direction is substantially constant.

In addition, there is an area in which the crystallinity increases substantially linearly from the fixed side surface of the sample toward the central portion in the thickness direction, and there is an area in which the crystallinity increases substantially linearly from the movable side surface of the sample toward the central portion in the thickness direction. In FIG. 5, the area in which the crystallinity increases substantially linearly from the fixed side surface of the sample toward the central portion in the thickness direction is illustrated as a fixed side surface area, and the area in which the crystallinity increases substantially linearly from the movable side surface of the sample toward the central portion in the thickness direction is illustrated as a movable side surface area. In addition, the area in which the crystallinity is substantially constant in the central portion in the thickness direction is illustrated as a core layer area. In addition, between the fixed side surface area and the core layer area, there is an area in which the increase rate of crystallinity gradually decreases from the fixed side surface area to the core layer area. This area is illustrated as a fixed side boundary area in FIG. 5. In addition, between the movable side surface area and the core layer area, there is an area in which the increase rate in crystallinity gradually decreases from the movable side surface area to the core layer area. This area is illustrated as a movable side boundary area in FIG. 5. In this manner, an area of the resin molded article along the thickness direction may be divided into five areas (the fixed side surface area, the fixed side boundary area, the core layer area, the movable side boundary area, and the movable side surface area).

In addition, as illustrated in FIG. 5, it can be seen that a change in crystallinity in the thickness direction in the fixed side surface area is smaller than a change in crystallinity in the thickness direction in the movable side surface area. In other words, the crystallinity in the fixed side surface area gradually changes, and the crystallinity in the movable side surface area abruptly changes. The temperature of the fixed side surface is 40° C. and the temperature of the movable side surface is 90° C. In other words, it can be estimated that smaller the change in crystallinity in the vicinity of a portion in contact with the mold, the lower the temperature of the contact mold.

In this manner, it is possible to obtain the existence of a crystallinity distribution or the tendency of a change in crystallinity depending on a region by actually measuring the crystallinity at each minute interval in the thickness direction of the sample.

After measuring the crystallinity for a plurality of samples, a correlation between the set temperature Tm and crystallinity of the mold was derived from an actually measured crystallinity of each sample and the set temperature Tm of the mold at the time of injection molding of the sample (to be exact, the set temperature of the mold, which was in contact with the surface, the crystallinity of which was actually measured, among the fixed type mold and the movable type mold). In this case, for example, a correlation equation (regression equation) may be derived by inputting a combination of the actually measured crystallinity of each sample and the set temperature Tm of the mold in contact with the surface, the crystallinity of which was actually measured at the time of injection molding the sample, to regression calculation software, and performing regression calculation.

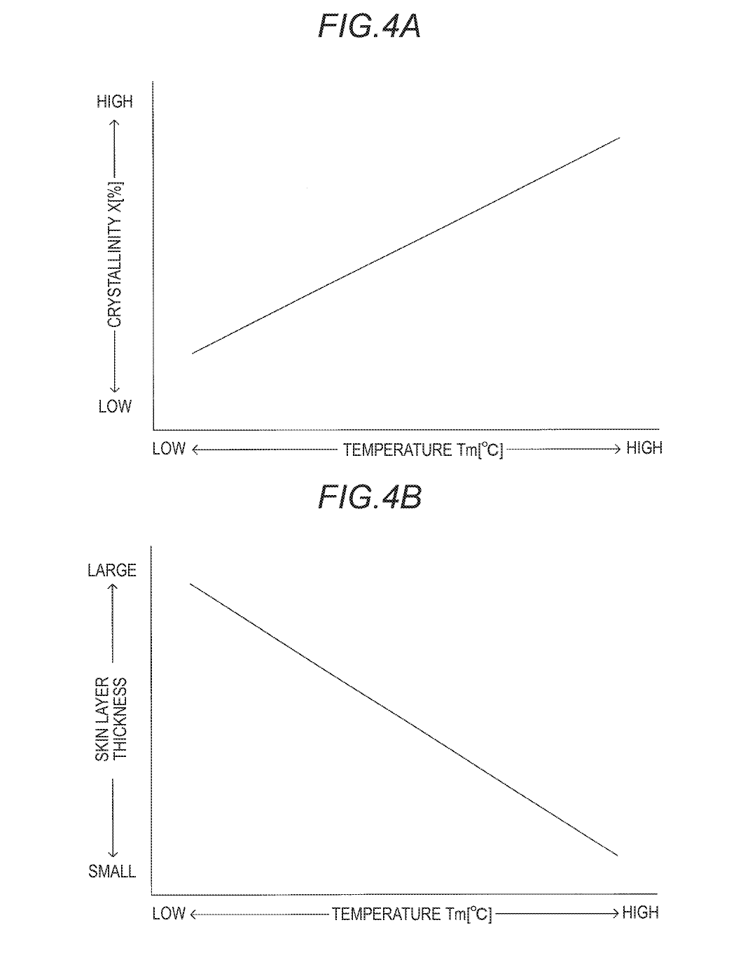

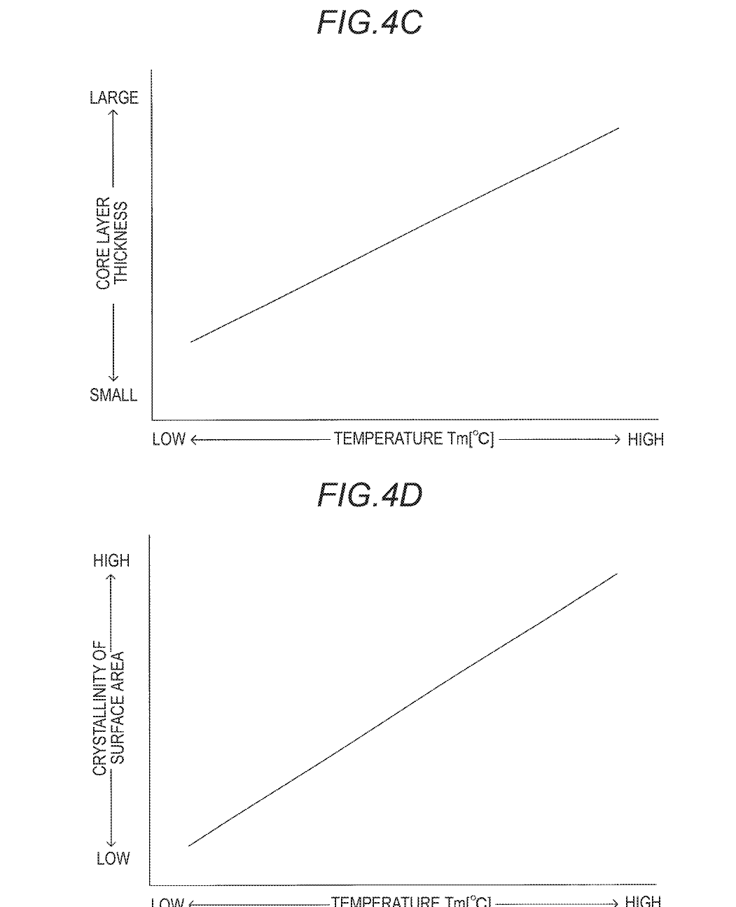

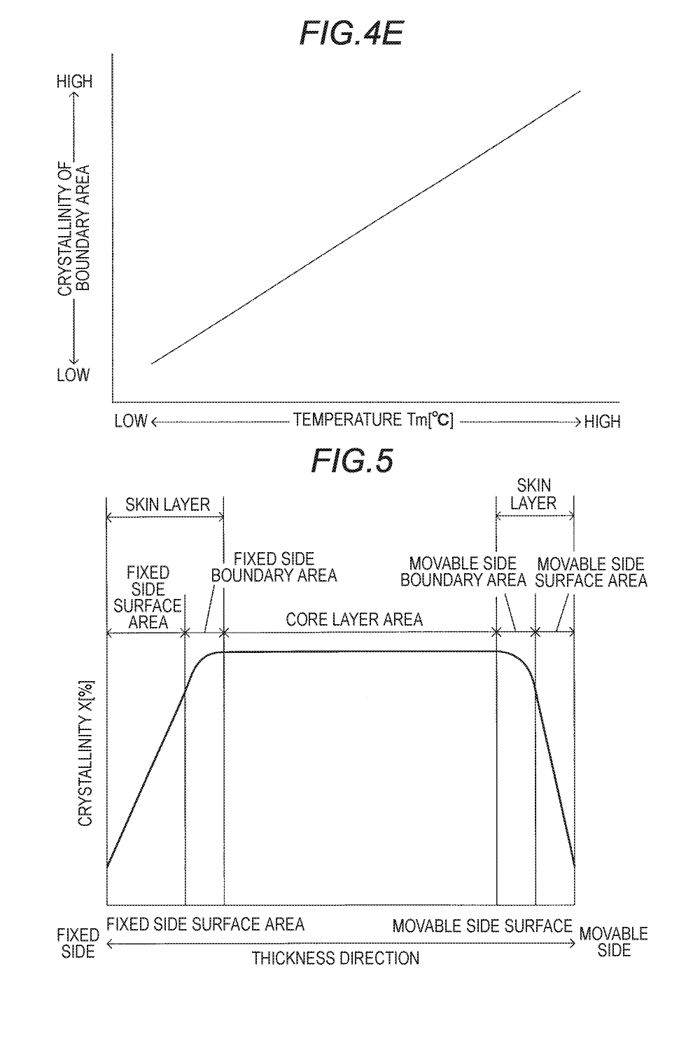

FIG. 4A is a graph illustrating a correlation between the crystallinity distribution and the mold temperature Tm. This graph is obtained from a correlation between the skin layer thickness and the mold temperature Tm illustrated in FIG. 4B, a correlation between the core layer thickness and the mold temperature Tm illustrated in FIG. 4C, a correlation between the crystallinity of the surface area and the mold temperature Tm illustrated in FIG. 4D, and a correlation between the crystallinity of a boundary area and the mold temperature Tm illustrated in FIG. 4E. It is possible to obtain the crystallinity (distribution) under each flow solidification condition from these correlations between the crystallinity distribution and the mold temperature Tm. That is, it is possible to obtain a correlation (first correlation) between the temperature change and crystallinity of the resin. Then, crystallization distribution data corresponding to the resin temperature distribution change (data) from the time of starting molding until the resin molded article is taken out from the mold is created by the obtained crystallinity (distribution). In addition, the correlation between the skin layer thickness and the mold temperature Tm (FIG. 4B), the correlation between the core layer thickness and the mold temperature Tm (FIG. 4C), the correlation between the crystallinity of the surface area and the mold temperature Tm (FIG. 4D), and the correlation between the crystallinity of the boundary area and the mold temperature (FIG. 4E) are obtained by actual measurement.

In the foregoing description, the “core layer thickness” refers to the thickness of a core layer in FIG. 5. In addition, the “skin layer thickness” refers to the sum of the thickness of the surface area and the thickness of the boundary area in FIG. 5 (in the example illustrated in FIG. 5, the skin layer thickness is the sum of the thickness of the fixed side surface area and the thickness of the fixed side boundary area, or the sum of the thickness of the movable side boundary area and the thickness of the movable side surface area).

### <Derivation of Relative Relationship (Second Relative Relationship) Between Crystallinity and Young's Modulus of Resin Molded Article>

Among the plurality of actually injection-molded samples as described above, an injection-molded sample is selected under a set temperature condition in which a difference between the set temperature of the movable type mold and the set temperature of the fixed type mold was the largest. For example, a sample, which is injection-molded under the set temperature condition of the mold in which the set temperature of the movable type mold was 90° C. and the set temperature of the fixed type mold was 40° C., is selected.

Subsequently, with regard to the selected samples, the Young's modulus at the measurement point of the crystallinity in the thickness direction was measured along the thickness direction at the interval of 10 μm. In the present embodiment, this measurement was performed by a nanoindentation method using a nanoindenter, but any other measurement apparatus capable of measuring the Young's modulus at a minute interval may be used. By this measurement, a Young's modulus distribution in the thickness direction may be obtained.

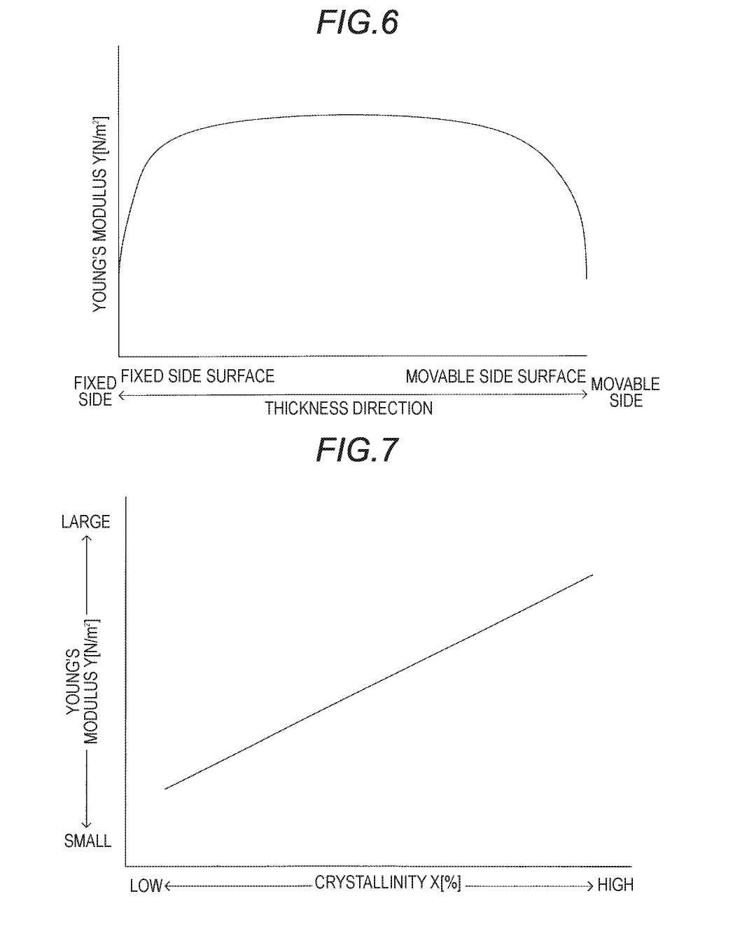

FIG. 6 is a graph schematically illustrating an actually measured Young's modulus distribution. In the graph of FIG. 6, the horizontal axis represents the position in the thickness direction of the sample, and the vertical axis represents the Young's modulus. In addition, in the graph of FIG. 6, the left end position of the horizontal axis is the fixed side surface, and the right end position is the movable side surface. As illustrated in FIG. 6, the Young's modulus also varies according to the position in the thickness direction of the sample, in the same manner as the crystallinity. That is, it can be seen that a Young's modulus distribution exists across the thickness direction of the sample. It can also be seen that the Young's modulus becomes higher from the surface toward the central portion in the thickness direction.

Subsequently, a correlation between the crystallinity and the Young's modulus was derived using the crystallinity distribution and the Young's modulus distribution actually measured at a minute interval (the interval of 10 μm) along the thickness direction of the sample. In this case, for example, a correlation equation (regression equation) may be derived by inputting a combination of the crystallinity and Young's modulus at the same measurement point to regression calculation software and performing regression calculation. FIG. 7 is a graph illustrating a correlation between the crystallinity X and the Young's modulus Y represented by the derived correlation equation. As illustrated in FIG. 7, it can be seen that there is a correlation between the crystallinity X and the Young's modulus Y in which higher the crystallinity X, larger the Young's modulus Y. In this way, a correlation between the crystallinity X and the Young's modulus Y of the resin molded article is derived. The derived correlation is stored in advance as the second correlation in the analysis device 3. Therefore, when executing the processing of S3 (Young's modulus distribution data creation step), the deformation analysis unit 60 calculates the Young's modulus corresponding to the crystallinity, which needs to be assigned to each mesh constituting a mesh division model of the resin molded article based on the first correlation, based on the second correlation, and assigns (sets) the calculated Young's modulus to the mesh. By assigning the Young's modulus corresponding to the crystallinity to each mesh in this manner, Young's modulus distribution data corresponding to crystallinity distribution data is created.

When the Young's modulus is assigned to each mesh in S3, a resin temperature distribution change, a linear expansion coefficient, and a Young's modulus are set for each mesh, respectively. That is, resin temperature distribution data, linear expansion coefficient distribution data, and Young's modulus distribution data are given to the mesh division model of the resin molded article.

Subsequently, in S4 of FIG. 2, the deformation analysis unit 60 calculates a deformation amount of the resin molded article when the surface temperature of the resin molded article is cooled down to room temperature based on the resin temperature distribution data, the linear expansion coefficient distribution data, and the Young's modulus distribution data given to the mesh division model of the resin molded article (deformation prediction step). Thus, deformation of the resin molded article is predicted. Then, the deformation analysis unit 60 predicts the deformed shape of the resin molded article based on the deformation amount calculated in S4, and outputs data indicating the predicted shape to the display device 4 (S5). Thus, the shape of the deformed resin molded article is displayed on the display device 4.

In this way, the deformation analysis unit 60 predicts deformation using the mesh division model reflecting data on distribution of the Young's modulus of the resin molded article. Therefore, it is possible to more accurately predict deformation, compared to a case where the Young's modulus is given as a fixed value.

### Example

A shape model of a resin molded article having the same flat plate shape as the sample was created. Next, a mesh division model of the resin molded article was created through the mesh division of the shape model.

Subsequently, by setting the temperature of a movable type mold to 90° C. and the temperature of a fixed type mold to 40° C., and setting a predetermined molding condition as an input condition, a mold cooling analysis by the mold cooling analysis unit 20, a filling analysis by the filling analysis unit 30, and a pressure holding/cooling analysis by the pressure holding/cooling analysis unit 40 were performed. Thus, a resin temperature and a linear expansion coefficient at the time of molding are given to each mesh constituting a mesh division model of the resin molded article. That is, resin temperature distribution data (resin temperature distribution change data) and linear expansion coefficient distribution data are given to the mesh division model of the resin molded article.

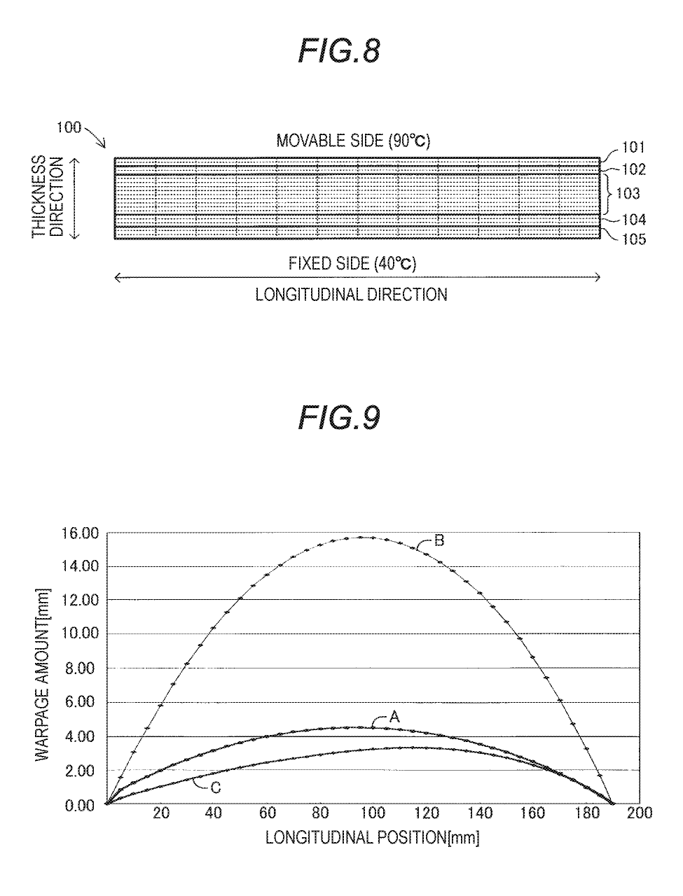

Subsequently, the mesh division model of the resin molded article was divided into five areas including a movable side surface area, a movable side boundary area, a core layer area, a fixed side boundary area, and a fixed side surface area along the thickness direction. FIG. 8 illustrates a state where a mesh division model 100 is divided into five areas. In the mesh division model 100 illustrated in FIG. 8, the horizontal direction is a longitudinal direction and the vertical direction is a thickness direction. As illustrated in FIG. 8, the mesh division model 100 is divided, in order from the upper side to the lower side in FIG. 8, into a movable side surface area 101, a movable side boundary area 102, a core layer area 103, a fixed side boundary area 104, and a fixed side surface area 105. Each of these areas corresponds to each area divided along the thickness direction based on the actually measured crystallinity illustrated in FIG. 5. Thus, the upper surface of the mesh division model illustrated in FIG. 8 is the surface that is in contact with the movable type mold having a temperature of 90° C., and the lower surface is the surface that is in contact with the fixed type mold having a temperature of 40° C.

In addition, each area is divided to have a thickness corresponding to the thickness of each area divided based on the actually measured crystallinity illustrated in FIG. 5. For example, in FIG. 5, it is assumed that the thickness of the sample is 2 mm, the thickness of the movable side surface area is 0.2 mm, the thickness of the movable side boundary area is 0.2 mm, the thickness of the core layer area is 1.0 mm, the thickness of the fixed side boundary area is 0.3 mm, and the thickness of the fixed side surface area is 0.3 mm. In this case, the ratio of the thickness of the movable side surface area 101 to the thickness of the sample is 10%, the ratio of the thickness of the movable side boundary area 102 to the thickness of the sample is 10%, the ratio of the thickness of the core layer area 103 to the thickness of the sample is 50%, the ratio of the thickness of the fixed side boundary area 104 to the thickness of the sample is 20%, and the ratio of the thickness of the fixed side surface area 105 to the thickness of the sample is 20%. Therefore, when dividing the mesh division model illustrated in FIG. 8 into the five areas, the mesh division model is divided into five areas, so that the rate occupied by each area matches the above-mentioned rate.

Subsequently, based on a correlation (first correlation) between the resin temperature (change) and the crystallinity obtained from the correlation between the mold temperature Tm and the crystallinity illustrated in FIG. 4A, the crystallinity corresponding to the resin temperature distribution data (resin temperature distribution change data) calculated by pressure holding/cooling analysis is obtained, and the obtained crystallinity is assigned to each area. Thereby, crystallinity distribution data is created. Thereafter, based on the correlation (second correlation) illustrated in FIG. 7, the Young's modulus corresponding to the crystallinity obtained for each area is obtained, and the obtained Young's modulus is set in each area. Thereby, Young's modulus distribution data is created, and the created Young's modulus distribution data is given to the mesh division model. In addition, in this case, the Young's modulus obtained for each area is assigned to all of the meshes constituting each area. Table 1 illustrates the Young's modulus set for each area.

TABLE 1

| Area | Young's modulus [N/m²] |
|---|---|
| Movable side surface area | 1.23 |
| Movable side boundary area | 2.31 |
| Core layer area | 2.52 |
| Fixed side boundary area | 2.39 |
| Fixed side surface area | 1.65 |

After setting the Young's modulus in each area in this manner, deformation (warpage) of the resin molded article at the time when the temperature of the resin molded article is cooled to room temperature was predicted by performing deformation calculation by the deformation analysis unit 60. In addition, for comparison, deformation (warpage) of the resin molded article was also predicted by performing the deformation analysis by the deformation analysis unit 60 even in the case where a constant Young's modulus (2.52 [N/m²]) was set for all of the meshes constituting the mesh division model of the resin molded article. In addition, the resin molded article having the same shape as the shape model was actually injection-molded under the same conditions as those described above. Then, the deformation amount (warpage amount) at the time when the injection-molded resin molded article was cooled to room temperature was actually measured.

FIG. 9 is a graph illustrating an actually measured warpage amount (deformation amount) and a predicted warpage amount (deformation amount). In FIG. 9, the horizontal axis represents the position in the longitudinal direction of the resin molded article (or the mesh division model), and the vertical axis represents the warpage amount (deformation amount) from the reference position. In addition, in FIG. 9, Graph A is a graph that illustrates a deformation amount predicted using a mesh division model to which a Young's modulus distribution is given (i.e. deformation prediction according to this example), Graph B is a graph that illustrates a deformation amount predicted using a mesh division model to which a constant Young's modulus is given for comparison (i.e. deformation prediction according to a comparative example), and Graph C illustrates an actually measured warpage amount (deformation amount) for an actually measured resin molded article.

As can be seen from FIG. 9, the predicted result of the deformation amount according to the comparative example (Graph B) is largely different from Graph C illustrating the actually measured warpage amount. On the other hand, the predicted result of the deformation amount according to this example (Graph A) is quite close to Graph C illustrating the actually measured warpage amount. From this, it can be seen that the accuracy of deformation prediction according to this example is high.

As described above, the method of predicting deformation of the resin molded article according to the present embodiment includes a resin temperature distribution data acquisition step S1 of acquiring resin temperature distribution data at the time of forming the resin molded article, a crystallinity distribution data creation step S2 of creating crystallinity distribution data, which is data on distribution of the crystallinity of the resin molded article corresponding to the resin temperature distribution data, based on the first correlation, which is a correlation between the temperature and crystallinity of the resin molded article, obtained using the actually measured crystallinity X of the resin molded article, which is actually injection-molded using the mold, a mechanical property value distribution data creation step S3 of creating Young's modulus distribution data (mechanical property value distribution data), which is data on distribution of the Young's modulus of the resin molded article corresponding to the crystallinity distribution data, based on the second correlation, which is a correlation between the crystallinity X and Young's modulus Y of the resin molded article, obtained from the actually measured crystallinity X and the Young's modulus Y (mechanical property value) of the resin molded article, which is actually injection-molded using the mold, and a deformation prediction step S4 of predicting the deformation of the resin molded article, which is taken out from the mold and is cooled to a predetermined temperature (for example, room temperature), by using the resin temperature distribution data and the Young's modulus distribution data.

According to the present embodiment, since the data on distribution of the Young's modulus as the mechanical property value of the resin molded article is given when predicting deformation of the resin molded article, prediction accuracy is improved compared to a case where the Young's modulus of the resin molded article is given as a fixed value in the prediction of deformation.

In addition, the Young's modulus distribution data of the resin molded article is derived based on the correlation between the crystallinity and the temperature obtained from the actually measured crystallinity and the correlation between the Young's modulus and the crystallinity obtained from the actually measured crystallinity and Young's modulus. Therefore, the actually measured value of Young's modulus is reflected in the Young's modulus distribution data. By using the Young's modulus distribution data reflecting the actually measured value, the accuracy of prediction of deformation of the resin molded article is further improved.

Although the embodiment disclosed here has been described above, this disclosure should not be limited to the above-described embodiment. For example, the resin to which this disclosure is applied is not limited so long as it is a crystalline resin. In addition, the resin to be used may be a fiber-reinforced resin containing reinforcing fibers, or may be a non-reinforced resin containing no reinforcing fiber. In addition, in the above embodiment, an example of creating data on distribution of the Young's modulus as a mechanical property value is illustrated, but data on distribution of other mechanical property values, for example, a modulus of transverse elasticity and a Poisson's ratio, may be created. In addition, the above embodiment illustrates an example in which the Young's modulus distribution data is created by dividing the mesh division model into five areas along the thickness direction and setting predicted values of the Young's modulus in each of the divided areas. However, without dividing the mesh division model into a plurality of areas, the Young's modulus may be set for each mesh based on the resin temperature given to each mesh, the first correlation, and the second correlation. In addition, in the above embodiment, the same Young's modulus is set in the plane direction (longitudinal direction and width direction) of the mesh division model, but, in a case where a temperature distribution exists in the plane direction, the Young's modulus corresponding to the resin temperature in the mesh may be set for each mesh divided in the plane direction. In addition, in the above-described embodiment, the synchrotron X-ray scattering method is used to actually measure the crystallinity distribution at a minute interval along the thickness direction of the sample, but the other methods (e.g., X-ray diffractometry, differential scanning calorimetry, infrared absorption spectroscopy, and Raman spectroscopy) may be used. In addition, in the above-described embodiment, the nanoindenter is used to actually measure the Young's modulus as a mechanical property value, but the mechanical property values may be actually measured using other devices such as, for example, a micro Vickers hardness meter, a scanning probe microscope. In addition, all of the steps described in the above embodiment may be executed by one piece of program software, or may be executed by using multiple pieces of program software. For example, only S3 of the respective steps of FIG. 3 illustrated in the above embodiment may be executed by separate program software.

The above-described embodiment has shown an example in which the first correlation is created using the set temperature of the mold as the resin temperature. Alternatively, the first correlation may be created using the mold temperature or the resin temperature (changing) in the molding process, which may be predicted by the mold cooling analysis unit 20, the filling analysis unit 30, and the pressure holding/cooling analysis unit 40. In addition, the crystallinity distribution data may be created using a correlation between the crystallinity and data (changing) on the pressure, the shear rate, or the like in the molding process, which may be predicted by the mold cooling analysis unit 20, the filling analysis unit 30, and the pressure holding/cooling analysis unit 40.

The above-described embodiment has shown an example in which the mesh division model is divided into five areas along the thickness direction, and the crystallinity and the mechanical property are assigned to each of the divided areas. Alternatively, the crystallinity and the mechanical property may be assigned to each of the elements (meshes, cells, or voxels) obtained by dividing the element division model in the thickness direction and the plane direction (longitudinal direction or width direction). These modified embodiments are useful measures to further improve the accuracy of prediction of the deformation amount of the resin-molded article. As described above, this disclosure may be modified without departing from the scope thereof.

An aspect of this disclosure provides a deformation prediction method of predicting deformation of a resin molded article, which is resin-molded using a mold, the method including: a resin temperature distribution data acquisition step (S1) of acquiring resin temperature distribution data at the time of forming the resin molded article; a crystallinity distribution data creation step (S2) of creating crystallinity distribution data, which is data on distribution of a crystallinity of the resin molded article corresponding to the resin temperature distribution data, based on a first correlation, which is a correlation between a temperature and crystallinity of the resin molded article and is obtained using an actually measured crystallinity of the resin molded article, which is actually resin-molded using the mold; a mechanical property value distribution data creation step (S3) of creating mechanical property value distribution data, which is data on distribution of a mechanical property value of the resin molded article corresponding to the crystallinity distribution data, based on a second correlation, which is a correlation between the crystallinity and the mechanical property value of the resin molded article and is obtained from the actually measured crystallinity and the mechanical property value of the resin molded article, which is actually resin-molded using the mold; and a deformation prediction step (S4) of predicting the deformation of the resin molded article, which is taken out from the mold and is cooled to a predetermined temperature, using the resin temperature distribution data and the mechanical property value distribution data.

According to the aspect of this disclosure, based on the correlation (first correlation) between the temperature and crystallinity of the resin molded article, which is obtained using the actually measured crystallinity, the crystallinity distribution data corresponding to the resin temperature distribution data at the time of forming the resin molded article is created. In addition, based on the correlation (second correlation) between the crystallinity and the mechanical property value obtained from the actually measured crystallinity and the mechanical property value, the mechanical property value distribution data corresponding to the crystallinity distribution data on the resin molded article is created. Thus, it is possible to derive the mechanical property value distribution data corresponding to the resin temperature distribution data from the two correlations. Then, the deformation of the resin molded article is predicted using the resin temperature distribution data and the mechanical property value distribution data. Since the mechanical property value distribution data on the resin molded article is given at the time of predicting the deformation of the resin molded article in this manner, prediction accuracy is improved, compared to a case where the mechanical property value of the resin molded article is given as a fixed value.

In addition, the mechanical property value distribution data on the resin molded article according to the aspect of this disclosure is derived based on the correlation between the crystallinity and the temperature obtained from the actually measured crystallinity and the correlation between the mechanical property value and the crystallinity obtained from the actually measured crystallinity and the mechanical property value. For this reason, the actually measured value is reflected in the data on the mechanical property value distribution. By using the data on distribution of the mechanical property values reflecting the actually measured value, the accuracy of prediction of the deformation of the resin molded article is improved, compared to a case of using the distribution of mechanical property values obtained from a theoretical equation.

As described above, according to the aspect of this disclosure, it is possible to provide a method of predicting deformation of a resin molded article, whereby the accuracy of prediction of the deformation is sufficiently improved.

The mechanical property value of the resin molded article may be one or more of a Young's modulus, a modulus of transverse elasticity, and a Poisson's ratio. These mechanical property values are particularly strongly involved in the deformation of the resin molded article. Therefore, by predicting the deformation of the resin molded article by using one or more of the data on distribution of these mechanical property values, it is possible to further improve the prediction accuracy. In addition, in the aspect of this disclosure, for example, a linear expansion coefficient or a shrinkage rate of the resin does not correspond to the mechanical property values.

The resin temperature distribution data at the time of molding may be resin temperature distribution change data, which is data indicating a change in a resin temperature distribution from the time of starting forming of the resin molded article to taking out of the resin molded article from the mold. By predicting the deformation of the resin molded article by using such data, it is possible to further improve the prediction accuracy. In addition, the resin temperature distribution change data may include resin temperature distribution data at the time of starting molding, resin temperature distribution data at the time of filling, resin temperature distribution data in a cooling process, and resin temperature distribution data at the time of taking out the resin molded article from the mold.

The crystallinity distribution data creation step may create the crystallinity distribution data corresponding to the resin temperature distribution data based on the resin temperature distribution data and the first correlation. According to this, the crystallinity corresponding to the resin temperature in a predetermined region of the resin molded article, which is indicated by the resin temperature distribution data on the resin molded article, is obtained from the first correlation. By obtaining the crystallinity corresponding to the resin temperature in each region of the resin molded article in this manner, it is possible to create the crystallinity distribution data on the resin molded article.

The mechanical property value distribution data creation step may create the mechanical property value distribution data corresponding to the crystallinity distribution data based on the crystallinity distribution data and the second correlation. According to this, the mechanical property value corresponding to the crystallinity in a predetermined region of the resin molded article, which is indicated by the crystallinity distribution data on the resin molded article, is obtained from the second correlation. By obtaining the mechanical property value corresponding to the crystallinity in each region of the resin molded article, it is possible to create the mechanical property value distribution data on the resin molded article.

The resin temperature distribution data may be created by assigning the resin temperature in a region corresponding to each of a plurality of elements constituting an element division model, which is created by dividing a shape model of the resin molded article into the plurality of elements, to each of the elements. The crystallinity distribution data may be created by assigning a crystallinity corresponding to the resin temperature, which is assigned to each of the plurality of elements constituting the element division model, to each of the elements, based on the first correlation. The mechanical property value distribution data may be created by assigning the mechanical property value corresponding to the crystallinity, which is assigned to each of the plurality of elements constituting the element division model, to each of the elements, based on the second correlation. According to this, an appropriate temperature and mechanical property value may be given to each element constituting the element division model of the resin molded article. Then, by predicting the deformation of the resin molded article using the element division model constituted by the elements to which the appropriate temperature and mechanical property value are given, it is possible to improve the prediction accuracy. The elements constituting the element division model may be, for example, meshes, cells, or voxels.

The first correlation may also be obtained based on the actually measured crystallinity of the resin molded article, which is actually resin-molded using the mold, and a temperature of the mold at the time of forming the resin molded article, the crystallinity of which is actually measured. Alternatively, the first correlation may be obtained based on the actually measured crystallinity of the resin molded article, which is actually resin-molded using the mold, and the resin temperature distribution data at the time of forming the resin molded article (the molding process), which is acquired in the resin temperature data acquisition step.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A deformation prediction method of predicting deformation of a resin molded article, which is resin-molded using a mold, the method comprising:
a resin temperature distribution data acquisition step of acquiring resin temperature distribution data at the time of forming the resin molded article;
a crystallinity distribution data creation step of creating crystallinity distribution data, which is data on distribution of a crystallinity of the resin molded article corresponding to the resin temperature distribution data, based on a first correlation, which is a correlation between a temperature and crystallinity of the resin molded article and is obtained using an actually measured crystallinity of the resin molded article, which is actually resin-molded using the mold;
a mechanical property value distribution data creation step of creating mechanical property value distribution data, which is data on distribution of a mechanical property value of the resin molded article corresponding to the crystallinity distribution data, based on a second correlation, which is a correlation between the crystallinity and the mechanical property value of the resin molded article and is obtained from the actually measured crystallinity and the mechanical property value of the resin molded article, which is actually resin-molded using the mold; and
a deformation prediction step of predicting the deformation of the resin molded article, which is taken out from the mold and is cooled to a predetermined temperature, using the resin temperature distribution data and the mechanical property value distribution data.

2. The method according to claim 1,
wherein the mechanical property value includes one or more of a Young's modulus, a modulus of transverse elasticity, and a Poisson's ratio.

3. The method according to claim 1,
wherein the resin temperature distribution data at the time of molding is resin temperature distribution change data, which is data indicating a change in a resin temperature distribution from the time of starting forming of the resin molded article to taking out of the resin molded article from the mold.

4. The method according to claim 1,
wherein the crystallinity distribution data creation step creates the crystallinity distribution data based on the resin temperature distribution data and the first correlation.

5. The method according to claim 1,
wherein the mechanical property value distribution data creation step creates the mechanical property value distribution data based on the crystallinity distribution data and the second correlation.

6. The method according to claim 1,
wherein the resin temperature distribution data is created by assigning the resin temperature in a region corresponding to each of a plurality of elements constituting an element division model, which is created by dividing a shape model of the resin molded article into the plurality of elements, to each of the elements,
the crystallinity distribution data is created by assigning a crystallinity corresponding to the resin temperature, which is assigned to each of the plurality of elements constituting the element division model, to each of the elements, based on the first correlation, and
the mechanical property value distribution data is created by assigning the mechanical property value corresponding to the crystallinity, which is assigned to each of the plurality of elements constituting the element division model, to each of the elements, based on the second correlation.
